(19) United States
(12) Patent Application Publication
(10) Pub. No.: US 2024/0111140 A1
(43) Pub. Date: Apr. 4, 2024

(54) DEVICE FOR CAPTURING MICROSCOPY IMAGES

(71) Applicant: PHIO SCIENTIFIC GMBH, Munich (DE)

(72) Inventor: Philipp PAULITSCHKE, Munich (DE)

(21) Appl. No.: 18/255,824

(22) PCT Filed: Dec. 3, 2021
(86) PCT No.: PCT/EP2021/084206

§ 371 Date: Jun. 2, 2023

(30) Foreign Application Priority Data
Dec. 4, 2020 (DE) 10 2020 132 308.5
Dec. 4, 2020 (DE) 10 2020 132 312.3

(51) Int. Cl. G02B 21/00; G01N 33/483; G02B 21/08; G02B 21/36

(52) U.S. Cl. CPC G02B 21/0008; G01N 33/4833; G02B 21/086; G02B 21/361; G02B 21/16

(57) ABSTRACT

The disclosure relates to devices for capturing microscopy images of a sample, comprising a partially coherent light source and an image sensor having a sensor head and sensor readout means. The sensor head and light source are arranged such that the sensor head and light source define a sample space therebetween for receiving a substrate having a sample, and such that an optical image of a sample in the sample space by the partially coherent light source onto the sensor head represents an intensity pattern. Further, the disclosure relates to systems for monitoring a sample having a plurality of such devices for capturing microscopy images.

### FIELD OF INVENTION

The present invention relates to the field of devices for capturing microscopy images, in particular lensless microscopes, and systems for monitoring samples, in particular biological cells.

### BACKGROUND OF THE INVENTION

Monitoring a sample with biological cells, especially recording and quantifying cell growth or mobility, is of great importance in research and development with biological cells. For example, the growth conditions in an experiment with living cells should be reproducible in cancer research and in a study in drug development, so that experiments or developments can be compared with each other.

In the context of research and development, biological cells are typically in a controlled environment, e.g., an incubation cabinet with limited volume, which has regulation of temperature, O2, CO2 environment/content, humidity, etc. Furthermore, a large number of cell samples are regularly cultured in parallel or used in cell-based experiments.

The monitoring of such cell cultures or the experiments with cell cultures are carried out, for example, on the basis of parameters such as cell growth, morphology, cell mobility, cell confluence. These parameters can be used to detect whether the desired cell state has already been reached or whether irregularities occur and remedial measures need to be taken, e.g. renewal of the culture medium, disposal due to infection, transfection of the cells, addition of an active agent or splitting of cells, or a combination of the foregoing.

Cell culture monitoring is performed manually on a regular basis, i.e. by opening the incubator, removing the cell culture, placing the cell culture under a microscope, visual inspection by an experienced laboratory staff member and documentation of the essential parameters.

Such monitoring has a number of disadvantages, including that the controlled growth conditions are disturbed by removal from the incubator, that studies are possible only at a few time points and are random, and that quantification of parameters is subject to subjective impressions by the laboratory staff, limiting reproducibility by different users.

There is a need for devices and systems for automated continuous monitoring of cell cultures that allow parallelization of cultivation or experiments in the incubator cabinets used, provide objective results, and can be used without negatively affecting cell culture conditions, especially temperature and environmental conditions.

With this in mind, it is the object of the present invention to provide improved systems for monitoring specimens, particularly biological cells, and devices for acquiring microscopy images, compared to the above and other disadvantages.

### DESCRIPTION OF THE INVENTION

The above object is addressed by devices and systems according to the independent claims. The dependent claims describe preferred embodiments.

In a first aspect, the invention relates to a device for capturing microscopy images. The device includes a partially coherent light source and an image sensor. The image sensor includes a sensor head and a sensor readout device, wherein the sensor head and the light source are spaced apart such that the sensor head and the light source define a sample space therebetween for receiving a substrate having a sample, hereinafter sample space.

Further, the sensor head and light source are arranged such that an optical image of a sample (e.g., the cells, cell clusters, spheroids, microcolonies, or the like) in the sample space through the partially coherent light source onto the sensor head represents an intensity pattern.

The sample may comprise objects or living beings which are optically transparent, semi-transparent or opaque and wherein each object/living being measures from 1 □m (micrometer), preferably in the range of 5 to 50 □m (micrometer). In particular, the sample may be biological cells, for example epithelial cells (e.g., HeLa cells), fibroblast cells (e.g., 3T3 cells), or carcinoma cell lines (e.g., HuH7 cells or A549 cells); or collections of cells (spheroids, clusters).

In some embodiments, the sensor readout device may be spaced from the sensor head. In this case, the distance between the components is such that the sample chamber for holding the sample does not heat up or is essentially not heated up by the operation of the components. Thus, the sample is not affected by the waste heat of the sensor readout device (in particular its electronics). Such an arrangement allows the sensor head to be operated in close proximity to biological cells without, for example, affecting the biological cells by waste heat from the sensor readout device. In conventional sensors, on the other hand, the sensor readout device and the sensor head are arranged adjacent to each other.

In another aspect of the present disclosure, the spacing of the sensor readout device and the sensor head may also be provided in an apparatus in which the light source is not necessarily a partially coherent light source (and the optical imaging by the light source onto the sensor head is not necessarily an intensity pattern).

In some embodiments, the partially coherent light source may be a light-emitting diode (LED). The partial coherence of the light source ensures on the one hand that—in contrast to a non-coherent light source—the optical imaging of the sample onto the sensor head represents an intensity pattern (which—in contrast to a coherent light source—does not have to be holographically reconstructed) and that—in comparison to a coherent light source—a facilitated and cheaper fabrication with more compact dimensions, non-invasive use (e.g. due to radiation exposure or phototoxicity) and lower heat generation is achieved. Depending on the embodiment, the device may comprise one or more light sources or different spectral spectra. Further, the light source may be an output of an optical fiber that conducts the light.

In some embodiments, the light source and sensor head are arranged to be set up for lensless imaging. In particular, they may be arranged to be set up for lensless microscopy, the sensor data of which does not require transformation or back-calculation, but can be used directly as image data. This saves computing power since no computational steps are required. In addition, the use of lensless imaging facilitates the achievement of stable image series even in long-term experiments, since no (reconstruction) focal plane is needed, for example, to holographically decode the image data. Furthermore, in contrast to in-line holography-based approaches, in which artifacts such as “twin images” have to be removed, multiple image series with different wavelengths or distance variants are not required per temporal measurement point. Thus, no additional light sources or mechanically traversable or movable components (e.g., to move the sample, sensor, or light source) are required for a single image. Lensless imaging thus enables a compact design so that the interior space of an incubator can be used efficiently, and a further reduction of heat input into the incubator.

Preferably, no lens is arranged between the light source and the image sensor for this purpose. For the purpose of describing the present invention, the term lens is intended to mean an optical component that allows light refraction and/or light focusing. Preferably, living cells, even if they could cause light refraction or light focusing, are not considered an optical component. Filters that allow selective transmission or reflection are not intended to be encompassed by the term “lens”.

In particular, in some embodiments, no lens is arranged between the light source and the sample chamber and/or between the sensor head and the sample chamber. Additionally or alternatively, no lens and/or no optical component affecting the optical coherence of the light from the light source is arranged in the sample chamber.

In lensless imaging, compact dimensions are made possible because the minimum height in these cases is not determined by a lens and, in particular, its focal length. Instead, a low overall height can be achieved. A low overall height is advantageous especially in view of the limited space available in incubator cabinets and in view of the desired parallelization of monitoring. Thus, a low overall height allows stacking of multiple devices according to the invention.

In some embodiments, the distance of the sensor readout device from the sensor head is such that the sample space is substantially not heated by the operation of the sensor readout device. Preferably, the heating of the sample space during operation of one (or more) devices is less than 1° C., preferably less than 0.1° C. For example, the distance may be more than 1 cm, preferably more than 5, 10 or 100 cm. This can be achieved, for example, by the spacing being such that the sensor head is adapted to be disposed in an incubator cabinet and the sensor readout device is adapted to be disposed outside the incubator cabinet. In other embodiments, the sensor readout device may be located within an incubator cabinet. In some embodiments, the distance may be predetermined based on the electrical output of the sensor.

Furthermore, the device can be set up so that the sensor readout device is switched on or off in a time sequence. For example, a multiplexer, MOSFET, switchable USB hub or relay can be provided as a digital circuit. In particular, the sensor readout device can be turned on in a clock sequence that corresponds to the desired monitoring frequency or sampling rate. Depending on the cell culture, a determination of key growth or mobility parameters may occur once per second, minute, hour, or day, or a multiple thereof. For example, monitoring can happen every minute, every two minutes, every five minutes, or every ten minutes. By selectively turning on the sensor readout device at specific times in this manner, heat input to the incubator is kept low. Thus, the sensor readout device does not generate heat during times when it is not capturing images or reading the image sensor. The same applies to the sensor head.

Further, the device may include passive cooling. For example, the sensor readout device may have a thermally conductive material on a side facing away from or toward the sensor head. In particular, the device may not include active cooling. This facilitates a compact design and reduces the occurrence of temperature gradients.

The intensity pattern represents a pattern of (local) intensity maxima and intensity minima. It can be, for example, a refraction, absorption, shadowing, diffraction or interference pattern caused by refraction at the sample, in particular caused by a single cell, which also acts as a (“living”) microlens. Preferably, samples can be used whose surface is curved and/or whose volume has a different refractive index than the surrounding medium.

The intensity pattern does not represent a real/photographically correct image of the sample, since optical assemblies such as imaging lenses are necessary for this. Compared to normal microscopic images, it rather appears to the human eye as an image with unusual intensity gradients, which, however,—compared to a holographic image—allows, among other things, a direct and unambiguous assignment of the cells even at higher cell densities and contains information such as cell morphology. A direct assignment to a real image is possible, because all information is included and a spatial delimitation to the background (usually areas without cells) is clearly possible. The optics and the evaluation algorithms are designed in such a way that a quantification of the relevant cell parameters can be made from them.

For this purpose, it is advantageous if the intensity pattern of a single object/a single cell has as few as possible, e.g. two, intensity extrema, for example a (local) intensity maximum and a (local) intensity minimum. The intensity extrema are also as spatially localized as possible. By an intensity maximum is meant, among other things, a region of constructive interference and/or increased intensity due to light focusing/refraction by a cell. An intensity minimum is, among other things, an area of destructive interference and correspondingly reduced intensity or the shading of the light by e.g. the cell(s).

This allows that on the one hand a high spatial contrast (by distinguishing between a local intensity maximum and a local intensity minimum) is achieved. On the other hand, the relatively low number of intensity extrema simultaneously reduces the image area affected by an object/cell, so that even at high object/cell densities the individual objects/cells remain identifiable.

The use of a partially coherent light source allows a single object (cell or cell cluster) to have as few (e.g., two) intensity extrema as possible while maintaining high contrast with the background.

In particular, a coherence length of the light source can be predetermined for this purpose in such a way that the intensity pattern of a sample (in particular a cell) in the sample space has one or more local intensity extrema. For example, the coherence length can be between 1 and 100 μm, preferably between 5 and 50 μm.

Alternatively or additionally, a spectral width of the light source may be predetermined such that the intensity pattern of a sample (in particular a cell) in the sample space has one or more local intensity extrema. For example, the spectral width may be between and 50 nm, preferably between 20 and 40 nm. The spectral width in this context is defined as Full Width Half Maximum (FWHM).

Alternatively or additionally, a divergence angle of the light source may be predetermined such that the intensity pattern of a sample in the sample space has one or more local intensity extrema. For example, the divergence angle can be between 0 and 5 degrees.

In some embodiments, the device may further comprise one or more filters disposed between the light source and the sensor head. For example, this may be one or more fluorescence filters, polarization filters, and/or neutral density filters. In particular, it may be bandpass, lowpass, or highpass filters. In particular, the filter may be directly attached (e.g., vapor-deposited) to the sensor head. Preferably, no filters that affect spatial and temporal coherence are arranged between the light source and the sensor.

Fluorescence filters, typically consisting of an excitation filter between light source and sample and an emission filter between sample and sensor head, can be used to capture (lensless) fluorescence images of the sample.

For example, a filter can be applied to the sensor head. For example, a vapor-deposited infrared filter can be used to keep the heat radiation from the sensor head into the sample chamber low. This is particularly advantageous when the device is of open design, i.e., complete enclosure of the device is not necessary. An open design allows for accelerated passive gas exchange (moisture, oxygen, carbon dioxide, etc.) and eliminates the need for active gas exchange devices such as fans. This facilitates a compact design and accelerates the heat exchange of the sample with the environment. Heating and temperature adjustment of the sample chamber is counteracted.

Furthermore, a vapor-deposited filter can minimize the influence of ambient light (e.g. when an incubator door is opened). Preferably, an ND filter can be used for this purpose.

In another aspect of the present disclosure, directly mounting a filter directly on the sensor head may also be realized in a device in which the light source is not necessarily a partially coherent light source (and the optical image through the light source to the sensor head is not necessarily an intensity pattern).

In some embodiments of the device, the sensor head is a CMOS sensor (CMOS here stands for Complementary Metal Oxide Semiconductor) or a CCD sensor. Although the sensor head may include a microlens for each pixel/photodiode, for example, such a combination of photodiode and microlens is also suitable for “lensless” imaging. Accordingly, the microlenses arranged directly on each photodiode are not covered by the prohibition of a lens arrangement between the light source and the sensor head. To this extent, the microlens is considered to be associated with the sensor head.

In some embodiments, the sample compartment is configured to receive a container for biological cells, particularly cell growth vials, petri dishes, or cell growth plates. The container may have one compartment for holding one cell culture or multiple compartments for holding multiple cell cultures.

The cultivation of biological cells or the performance of experiments with cells can be particularly temperature-sensitive. This already starts with the settling (or sedimentation) of the cells, i.e., for example, before they adhere. Temperature gradients can lead to convection currents, so that sedimentation is strongly affected and its uniformity is impaired. In addition, during subsequent growth of the cells in the container, thermal or temperature gradients can alter migration, division behavior or nutrient supply.

With this in mind, in some embodiments, the device may further comprise a support element configured to support a sample (e.g., a container of biological cells) on a support surface of the support element and to receive the sensor head. This allows a constant heat distribution over the support surface. For this purpose, in particular the heat capacity and/or the thermal conductivity and/or the emissivity of the support element can be predetermined in such a way that it is equal or homogeneous over the entire contact surface with the sample container.

For example, the support element may comprise a first sub-region for receiving the sensor head and may comprise a second sub-region. The thermal conductivity (k in W/(m*K)) and/or heat capacity (C in J/K) and/or emissivity (ε in %) of the second sub-region may be determined to be substantially identical to the thermal conductivity or heat capacity of the first sub-region after receiving the sensor head. Living cells or other samples are affected by temperature differences. If the sensor head is warmer or colder relative to the sample, the cells will either be collected above it, or displaced. A substantially identical thermal conductivity or heat capacity will cause the sample to warm uniformly over the entire sample (e.g., when the cooler sample is placed in the incubator) or not warm at all (e.g., when generating images), thus avoiding or reducing such collection or displacement effects.

The support surface can also be set up for positioning the container, for example by having a profile that is adapted to the shape (e.g. the contours or the base) of the container. The adaptation is designed to position the container to within millimeters, preferably within 100 μm. Thus, a reproducible and permanent positioning can be achieved by simply placing the container on the support surface. For example, the sample container can be returned to the same position relative to the sensor head after removal. Furthermore, slippage over the duration of a long-term experiment is reduced. This also enables a compact arrangement of the devices, such as stacking.

In another aspect of the present disclosure, the aforementioned support element may also be provided in a device in which the light source is not necessarily a partially coherent light source (and the optical image through the light source to the sensor head is not necessarily an intensity pattern).

In addition, the devices may also be equipped with interlocking elements (such as groove/springs) and/or magnetic elements to allow the devices themselves to be placed above or next to each other without the possibility of them shifting relative to each other or falling away from each other.

In another aspect of the present disclosure, the spacing of the sensor readout device and the sensor head may also be realized in a device in which the light source is not necessarily a partially coherent light source (and the optical imaging by the light source onto the sensor head is not necessarily an intensity pattern).

In some embodiments, the sensor head and/or light source may be movable relative to the sample space for receiving the substrate and/or relative to the light source. In particular, it or they may be movable in a plane substantially parallel to the substrate to be received (and/or substantially perpendicular to the direction of propagation of light from the light source). This allows multiple containers to be inserted into the depth of the incubator in a confined space. Motorization of the sensor head (or even the container) is also possible, allowing a sample to be scanned to monitor a cell culture at different locations in the cell culture. This allows, for example, monitoring of the homogeneity of a cell culture.

Alternatively, or in addition, the scanning allows a container containing multiple compartments (e.g., a multi-well container) with multiple different cell cultures or measurement concentrations, e.g., drug concentrations, to be scanned. Thus, a single device can be used to monitor several different cell cultures or cell experiments. This serves to reduce the number of components that could cause heat input in an incubator and to increase compactness (by, for example, reducing a travel axis).

Such an arrangement and traversability allows a high degree of parallelization in a small space and already with a single device according to the invention. By using a single device for monitoring multiple cell cultures, the heat input of the monitoring device into the incubator is kept low.

Alternatively or additionally, the sample can be movable relative to the light source. This allows easier access of a user to the sample (e.g., for the exchange of a nutrient medium or the addition of an active ingredient), without spatial interference of the user interaction by the light source.

In some embodiments, the sensor may be movable with the sample so that the relative positioning of the sample to the sensor remains substantially unchanged by user interaction with the sample.

In some embodiments, the device may be adapted to be placed on top of another device (according to the invention) such that the former device cannot be displaced relative to or fall off of the further device.

In another aspect of the present disclosure, the aforementioned features of mobility and/or traversability and/or stackability may also be realized in a device in which the light source is not necessarily a partially coherent light source (and the optical image through the light source to the sensor head is not necessarily an intensity pattern).

In another aspect, the present invention relates to a system for monitoring a sample, in particular for monitoring biological cells. The system comprises one or more devices according to the invention. The system is adapted to allow a plurality of samples to be monitored by the one or more devices. This may provide further parallelization of monitoring. For example, a system may comprise at least two devices, at least three devices, at least ten devices, or at least one hundred devices.

In some embodiments, the system further comprises an evaluation device. The evaluation device may be a processor, a computer, a smartphone, a server, or an Internet interface.

In some embodiments, the system further comprises a drive device configured to turn the plurality of devices on or off in a timed sequence. For this purpose, a control device may comprise, for example, a multiplexer. This allows multiple sensor heads to be read in parallel or serially.

In some embodiments of the system, the sample chamber is configured to receive a multi-well container having multiple compartments. The devices of the invention are arranged in a pattern that is predetermined such that each compartment of the multi-well container can be imaged by one or more of the devices.

In particular, the plurality of samples may be arranged in the one multi-well container. In this case, the number (and arrangement) of compartments of the multi-well container may correspond to the number (and arrangement) of devices of the system. Alternatively, one movable device (relative to the compartments) may be provided for each column (or each row) of the multi-well bin, such that each device scans a corresponding column (or row).

In some embodiments, a system according to the invention comprises multiple devices arranged in a row (one-dimensional pattern). Other embodiments include devices arranged in a plane as a two-dimensional pattern. Still other embodiments include devices arranged in a three-dimensional pattern. Further, a system, for example, having a two-dimensional pattern of devices, may be adapted to allow multiple such systems to be stacked. Such systems (or devices) may be referred to as stackable. Hereby, further parallelization can be achieved while consuming little space. This parallelization is facilitated by the very low heat generation mentioned above. Even with a large number of closely adjacent (e.g. stacked) fixtures, the heat input into an incubator is reduced. The optically robust design without the use of lenses or the like also contributes to the fact that the devices can be stacked and the samples can be manipulated if necessary, for example to exchange a culture medium or to add an active ingredient.

Further, in some embodiments of the system, the side-by-side and/or stacked devices may be in physical and/or electrical and/or magnetic contact with each other.

Devices or systems according to the invention may further comprise one or more drawers. These drawers are arranged to receive a container (e.g., a cell culture dish, a cell culture bottle, or a multi-well plate) and to move it into or out of the sample compartment.

In particular, the system or at least the light source(s) and sensor head(s) of the respective devices according to the invention is adapted to be arranged in an incubator. In particular, any sensor evaluation device and/or the sensor readout devices may be arranged inside or outside the incubator.

In another aspect, the present invention relates to the use of a device or system according to the invention for monitoring a sample, in particular biological cells. For example, monitoring may comprise moving the device of the system and/or moving the samples.

### BRIEF DESCRIPTION OF THE FIGURES

In the following description of embodiments of the invention, reference is made to the accompanying drawings, which show:

FIG. 1 a schematic diagram of a device for lensless microscopy;

FIG. 2A a schematic representation of a device according to one embodiment;

FIG. 2B a schematic representation of a device according to a further embodiment;

FIG. 3A An example of an illustration showing a device according to the invention;

FIG. 3B an example of an illustration with a known device;

FIG. 4 a schematic diagram of a sensor of a device according to one embodiment;

FIG. 5 aschematic representation of sensors of a system according to one embodiment;

FIG. 6 a schematic representation of a system according to one embodiment;

FIG. 7 a schematic representation of a system according to one embodiment;

FIG. 8 a schematic representation of a device according to one embodiment;

FIG. 9 a schematic representation of a system according to one embodiment;

### DESCRIPTION OF EXEMPLARY EMBODIMENTS

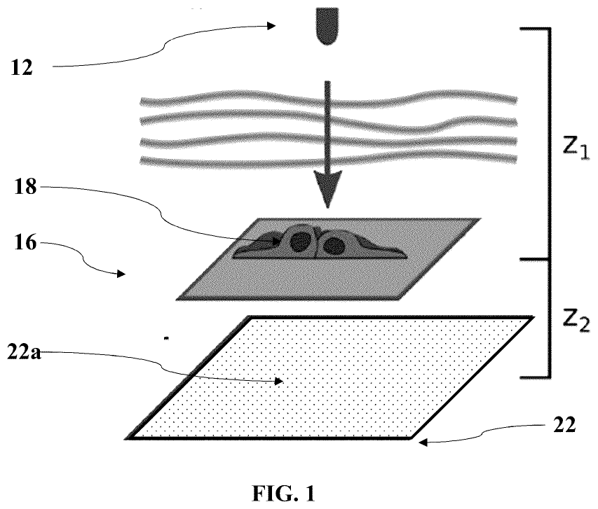

FIG. 1 shows a schematic diagram of a device for lensless microscopy. The device includes a light-emitting diode 12 (LED), as a light source, and a sensor head 22, which is based on CMOS technology.

The sample 16 is located at a distance z1 from the LED 12. Preferably, the distance z1 is predetermined by the thickness of a substrate carrier or a container with cells, e.g. a Petri dish. Alternatively, the container may be multi-well plates or cell culture vials. Accordingly, the distance z1 is preferably a few cm, particularly preferably between 1 and 10 CM.

A portion of the partially coherent light emitted by the LED (represented by the partially parallel wavefronts), which propagates substantially in the direction of the sample (represented by the vertical arrow in FIG. 1), is scattered by the sample 16 with biological cells 18 adhered to a substrate. Another portion of the light passes through the sample 16 substantially undisturbed, i.e., unscattered.

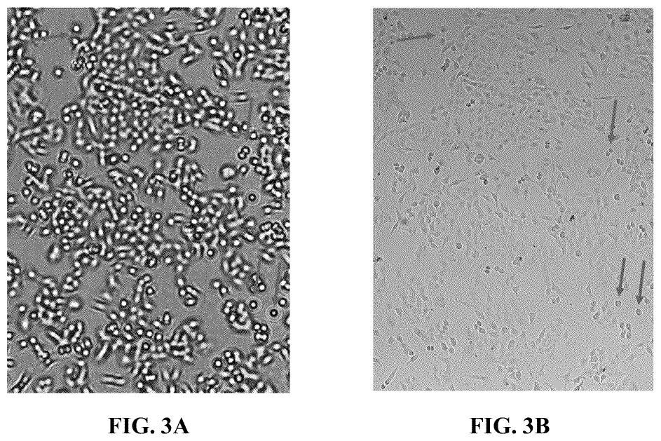

The scattered light waves are refracted and/or interfere with the unscattered light waves, resulting in an optical image of the sample 16 as an intensity pattern 22a, which is recorded by the CMOS sensor head 22. An exemplary intensity pattern is shown in FIG. 3A. The sensor head 22 is located at a distance z2 from the cell sample 16. Preferably, the distance z2 is less than 1 cm, more preferably between 0.1 and 3 mm.

The partial coherence is significantly determined by the spatial extent of the light source and by the spectral width of the light source. The spatial extent of the light source can be selected directly by choosing an appropriate LED or indirectly by using an optical fiber. The choice of a spatial extent can be determined in particular by the choice of a divergence angle of the light source.

Alternatively or additionally, a narrow spectral width, e.g. in the range 5-50 nm, can be selected.

The device shown in FIG. 1 does not have a lens. Accordingly, the partial coherence of the light used results in an intensity pattern.

The image captured by means of the sensor head can be used in a method for monitoring the cells. For this purpose, the image acquired as an intensity pattern can be transformed into, or at least interpreted as, an image that is substantially equivalent to the image obtained with a conventional lens-based microscope. This can be achieved, for example, by means of an algorithm that has been trained, for example, by machine learning. Alternatively, the acquired image can be evaluated directly without any further transformation or computation step, for example to determine parameters that allow monitoring of the cell culture. For example, cell detection or cell tracking can be used to monitor the cell culture. The parameters to be determined include in particular parameters describing cell confluence, morphology, motility, division or vitality state (alive/dead).

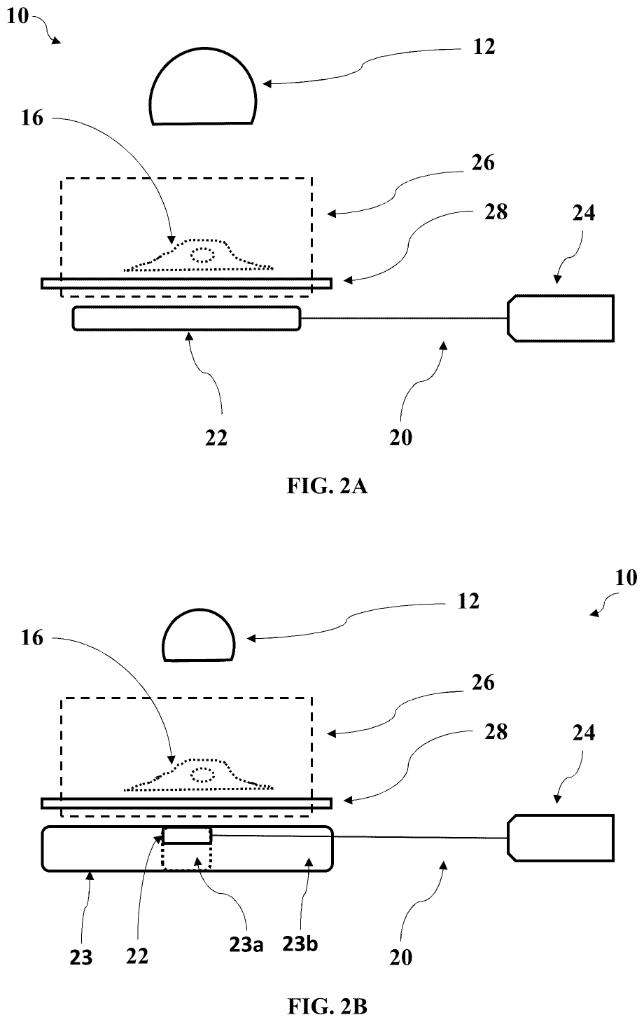

FIG. 2A shows a schematic diagram of a device 10 for capturing microscopy images of a sample 16. The device includes a partially coherent light source 12 and an image sensor 20. The light source 12 is a light-emitting diode (LED).

The image sensor 20 includes a sensor head 22 and a sensor readout device 24, the sensor head and the light source 12 being spaced apart such that the sensor head 22 and the light source 12 define a sample space 26 therebetween for receiving a substrate 28 having a sample 16, hereinafter sample space 26.

Further, the sensor head 22 and the light source 12 are arranged such that an optical image of the sample 16 through the light source 12 onto the sensor head 22 represents an intensity pattern.

In addition, the heat-generating electrical assemblies on the side facing away from the cell are in thermal contact with the remaining assemblies in order to dissipate the heat. This can be achieved by means of potting material.

Heat-generating processes (such as image conversion) take place as far away as possible from the sensor head and thus also from the sample. The use of a MIPI-compatible sensor and an associated data pipeline enables the optimization of the thermal behavior of the sensor head, for example by means of PCB (printed circuit board) technology or sensor PowerDown.

In addition, the sensor readout device 24 is spaced apart from the sensor head 22 so that the sample chamber 26 is substantially not heated by the operation of the sensor readout device 24. The spacing is predetermined as a function of the electrical output of the sensor. The spacing may also be such that the sensor readout device 24 may be located outside of an incubator in which the other components of the device 10 are located.

Accordingly, the biological cells 16 are not affected by waste heat from the sensor readout device 24 even though the sensor head 22 is operated in close proximity to the biological cells.

The LED 12 and the sensor head 22 are arranged to allow lensless imaging. In particular, no lens or other optical component that allows light refraction and/or light focusing is arranged between the light source and the image sensor. This allows for a particularly compact design. The distance between sensor head 22 and sample 16 is preferably less than 1 cm, particularly preferably between 500 and 2000 Om (micrometers). It is therefore a recording that does not take place in the far field. This relatively small distance is also advantageous in that it keeps the image area influenced by an object/cell small. At larger distances (between sensor and sample), the intensity patterns of neighboring objects/cells overlap.

Furthermore, the absence of lenses or the like allows robust operation, even for long-term experiments: This omission minimizes the susceptibility or risk of “drifting” (such as a focal plane) that regularly occurs with lens-based microscopes.

FIG. 2B shows a schematic representation of a further device 10 for capturing microscopy images of a sample 16. The device 10 according to FIG. 2B is identical in essential parts to the device according to FIG. 2A. It further comprises a support element 23. The support element 23 is adapted, on the one hand, to support the substrate 28 with the sample 16. For this purpose, it has a support surface.

In addition, the support element 23 is designed to accommodate the sensor head 22. For this purpose, the support element in the example shown has a recess which is dimensioned to accommodate the sensor head. The support element thus has a smaller thickness at the recess than at other parts of the support element. By receiving the sensor head in the recess, the recess is filled in such a way that a substantially flush surface is formed which serves as a support surface.

The support element is designed to have a thermal capacity and a thermal conductivity of the support element (with the sensor head received) that is substantially homogeneous over the support surface, i.e., the contact surface with the substrate 28. This can be achieved, for example, by the support element comprising a plurality of sub-regions having different materials.

A first partial region may be formed, for example, by the above-mentioned region of reduced thickness at the recess for the sensor head. The thermal conductivity and heat capacity of this first sub-region 23a may be reduced, for example, by selecting a material having relatively low thermal conductivity and low heat capacity and low emissivity (in particular, a thermal insulator, such as Plexiglas or porous materials having a high air content). This can offset or compensate for the relatively greater thermal conductivity and thermal capacity and emissivity of the sensor head (whose material is essentially, for example, a metal/semiconductor/insulator composition).

A second sub-region 23b of the support element may be formed, for example, by the above-mentioned region(s) of non-reduced thickness, i.e., around the recess. The thermal conductivity and thermal capacity of this second sub-region 23b may be selected to be approximately equal to the combined thermal conductivity and thermal capacity of the first sub-region 23a with sensor head 22 received in the recess.

A substantially homogeneous thermal conductivity and thermal capacity of the support element over the support surface allows any temperature differences (in particular between the support surface on the one hand and substrate/sample, for example when inserting a sample into the device 10 which is already in an incubator) to be uniformly dissipated, avoiding or reducing (e.g. to below 1° C., preferably to 0.1° C.) temperature gradients along the support surface. Avoiding or reducing temperature gradients along the support surface minimizes the risk of convection currents, which can lead to inhomogeneities in the sample. Such convection currents can, for example, cause sedimentation (settling) of biological cells to occur unevenly as the cells flow in the direction of the temperature gradient along the support surface due to the convection currents. The materials are selected in such a way that no or essentially no convection currents occur during the thermalization of the sample with the environment, which can regularly occur in any case before operation of the device (e.g. temperature equalization between cell medium incl. biological cells and the incubator environment), which cause a redistribution of the cells.

FIG. 3A shows an example of an image taken with a lens-less microscope. The intensity pattern does not represent a photographically correct image of the sample. Despite the low profile of the device used, the image exhibits a wide field of view and is essentially limited by the size of the sensor head, but not by any (nonexistent) optical imaging using lenses. In particular, an intensity pattern can be seen which represents an imaging of the cells onto the sensor head.

In particular, the intensity pattern has an area of high intensity (light) in the center of the cells. Around this area of high intensity is shown an area of low intensity (dark), which not only has a lower intensity than the center (intensity maximum), but also a lower intensity than the background (gray).

In contrast, FIG. 3B shows an example of an image of the same sample taken with a lens-based microscope. The lens-based microscope also allows monitoring of parameters describing cell confluence, motility, or division. However, it requires a larger height and more complex fabrication in comparison. In addition, the spacing of the optical components must be manually adjusted to obtain a “sharp” image. Furthermore, non-invasive observation over a longer period of time (e.g. several days) is more difficult, insofar as “drifting” of the optical components can lead to defocusing.

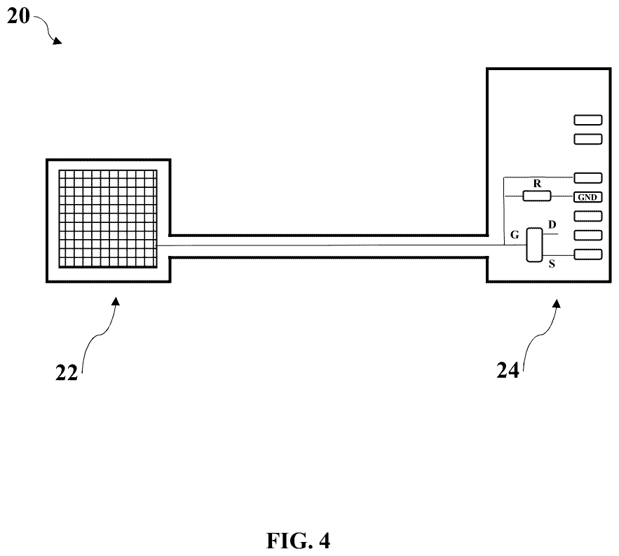

FIG. 4 shows a schematic representation of an image sensor 20, such as may be used in the device 10 shown in FIG. 2A or shown in FIG. 2B. The image sensor 20 includes a sensor head 22 and a sensor readout device 24.

The sensor readout device 24 is arranged at a distance from the sensor head 22. In the case shown, the distance is a multiple of the size of the sensor readout device 24 and the sensor head 22, in this case approximately a distance of 1-2 cm. Such an arrangement enables the sensor head to be operated in the immediate vicinity of biological cells without affecting the biological cells by waste heat from the sensor readout device.

The image sensor 20 is a CMOS sensor and has a photoactive raster plate whose photosensitive pixel elements provide spatial resolution for imaging irradiance. Photodiodes are used as pixel elements, which are assembled in a known manner to form a semiconductor detector in CMOS manufacturing.

The sensor readout device comprises electronic circuits which are connected to the sensor head in such a way that they read out signals from the individual pixel elements and combine them to form an image.

The sensor readout device 24 is arranged to be turned on or off in a timed sequence. For this purpose, the circuit device can be controlled by a drive device (e.g. a multiplexer, MOSFET, switchable USB hub or relay). Thus, the sensor readout device can be turned on in a clock sequence that corresponds to the desired monitoring frequency or sampling rate. Depending on the cell culture, a determination of key growth or mobility parameters can occur once per second, minute, hour, or day, or a multiple thereof. For example, monitoring can happen every minute, every two minutes, every five minutes, or every ten minutes. By such selective switching on of the sensor readout device at specific times, the heat input into the incubator is kept low.

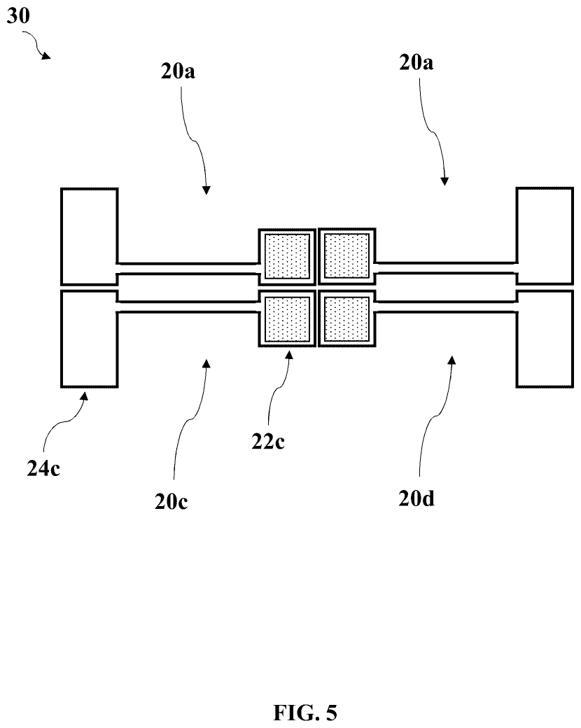

FIG. 5 shows a schematic representation of several sensors 20a-20d. These may be part of a system according to the invention. The four sensors are arranged in a 2×2-pattern. Other numbers of sensors and other patterns are also possible.

Each of the sensors 20a-20d has a sensor head and a sensor readout device. For example, the image sensor 20c has a sensor head 22c and a sensor readout device 24c.

In other embodiments, multiple sensors may share a common sensor readout device.

In any case, the sensor readout device(s) is/are spaced apart from the sensor heads so that none of the sensor readout device(s) heats the environment of any of the sensor heads and substantially does not heat the sample space (and thus the sample being observed).

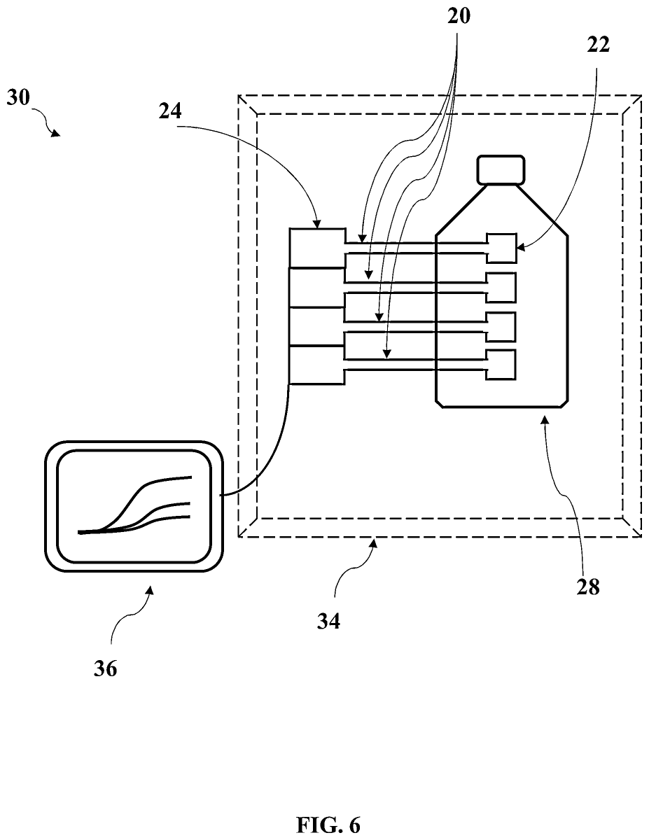

FIG. 6 shows a schematic diagram of a system 30. The system 30 includes a plurality of devices for capturing microscopy images, each having an image sensor 20. A total of four sensors 20 are shown. Each of the sensors 20 includes a sensor head 22 and a sensor readout device 24.

The four sensors 20 are arranged in a 4×1 pattern to monitor four different areas of a sample. In the case shown, the sample is in a cell culture bottle 28.

Furthermore, the system 30 comprises an evaluation device 36. In contrast to the sensors 20 and the sample bottle 28, the evaluation device 36 is arranged outside a cell culture incubator 34. This further minimizes the heat input into the incubator by the evaluation device. In addition, the arrangement of the evaluation device outside the incubator allows the monitoring results to be read without opening the incubator.

In the case shown, the evaluation device is a computer. Alternatively, the evaluation device can comprise a processor, a smartphone, a touch screen, a server or an Internet interface. In particular, the evaluation may take place locally and on a remotely located server or in a server cloud.

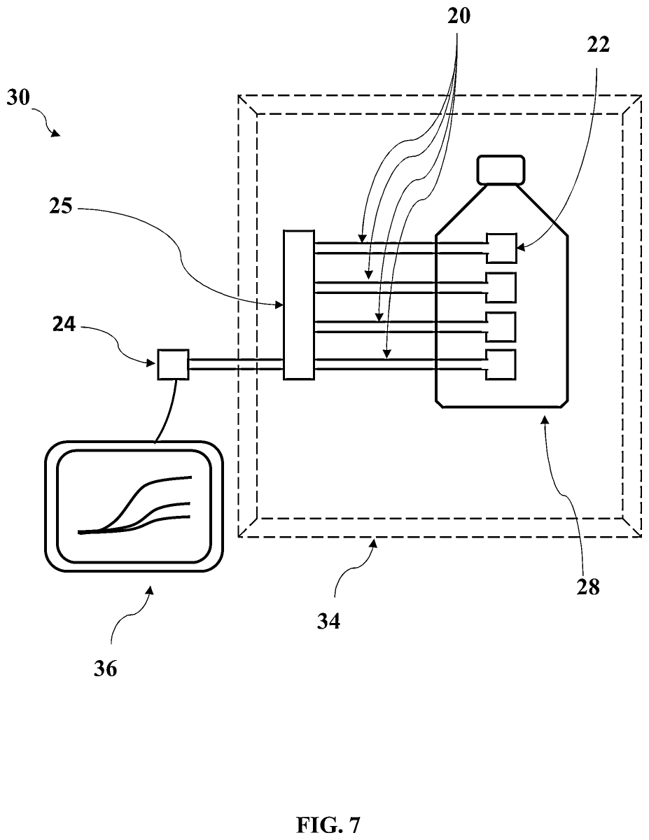

FIG. 7 shows a schematic representation of a system 30 that is substantially identical to the system according to FIG. 6. The system 30 according to FIG. 7 also comprises a plurality of devices, in particular a plurality of sensor heads 22. However, in contrast to FIG. 6, these plurality of sensor heads 22 are connected to a (single) sensor readout device 24 via a multiplexer 25. This single sensor readout device 24 serves as the sensor readout device for each of the sensor heads 22, and the multiplexer is designed to suitably transmit the respective signals from the plurality of sensor heads 22 to the sensor readout device 24. In the embodiment shown, the multiplexer is adapted to be located inside the incubator so that a single cable connection can be routed from inside the incubator to the sensor readout device located outside the incubator. In other embodiments, the multiplexer may be arranged outside the incubator to further reduce heat input.

In addition, the multiplexer or sensor readout device can serve as a control device that is set up to switch the multiple sensor heads on or off in a time sequence. This allows multiple sensor heads to be read out in parallel or serially. In particular, the sensor heads can be switched in a clock sequence that corresponds to the desired monitoring frequency or sampling rate. Such selective switching on of the sensor heads at specific times keeps the heat input into the incubator low.

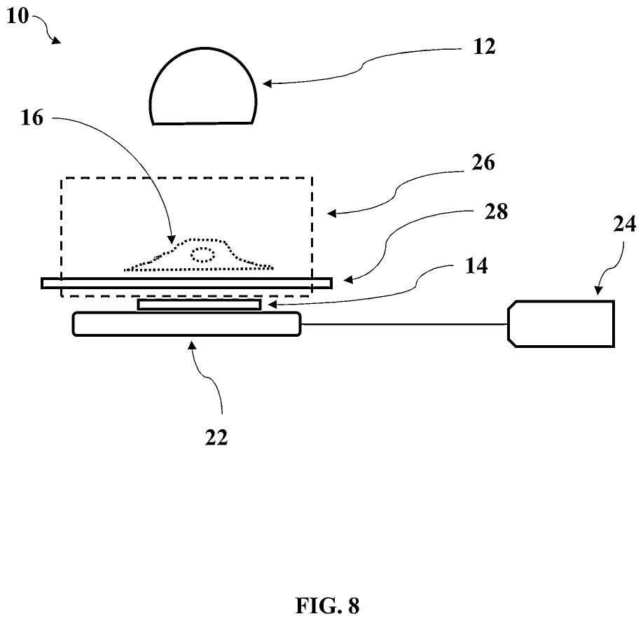

FIG. 8 shows a schematic representation of a device according to one embodiment. The device shown is similar in essential parts to the device shown in FIG. 2A.

Additionally, the device 10 of FIG. 8 includes a filter 14 disposed between the sensor head 22 and the sample 16. The filter 14 is an infrared filter for minimizing thermal radiation from the sensor head to the sample. The filter can be vapor deposited onto the sensor head, for example.

In other embodiments, the filter may be a fluorescence emission filter. In these cases, another filter, a fluorescence excitation filter, may be disposed between the LED and the sample 16.

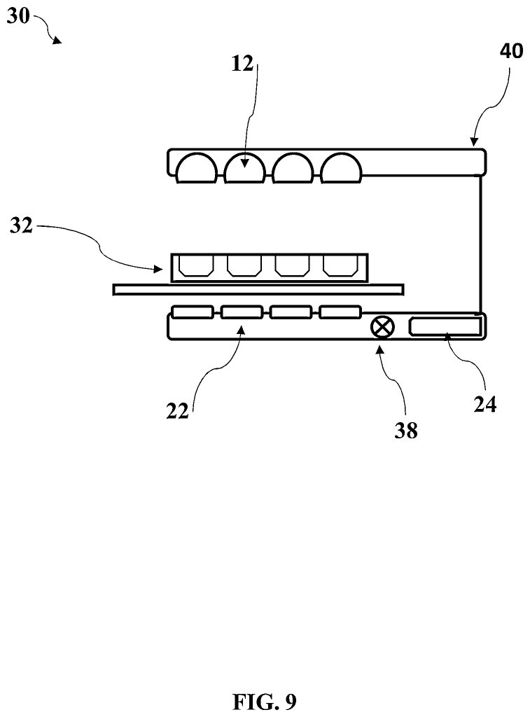

FIG. 9 shows a schematic diagram of a system 30 according to another embodiment.

The system 30 comprises four devices according to the invention for recording microscopy, arranged in a 4×1 pattern. Each of these devices includes a light source 12 and a sensor head 22. The light sources and the sensor heads are connected to each other by means of a housing 40 such that they can be moved together along an axis. The housing further includes a common sensor readout device 24, which in other embodiments may be located outside the incubator.

The sample chamber is configured to receive a multi-well container 32 having multiple compartments. In the case shown, a 36-well plate, with 36 compartments in a 4×9 pattern, is located in the sample compartment for monitoring.

The four fixtures are arranged in a 4×1 pattern so that each compartment of the multi-well container can be imaged by one of the fixtures.

The interconnected light sources 12 and sensor heads 22 are movable relative to the sample compartment for receiving the substrate, and thus relative to the multi-well-plate 32. In particular, they are movable in a plane substantially parallel to the substrate to be received. In the present case, they are movable along an axis such that the multi-well-plate 32 is scanned to monitor each of the compartments. For this purpose, the system includes a linear motor 38.

In other embodiments, a system according to the invention may include multiple motors for traversability in multiple dimensions. For example, a single device for capturing microscopy images (i.e., with one sensor) may be used to scan a multi-well plate.

1. Device for capturing microscopy images of a sample, comprising:
a partially coherent light source; and
an image sensor including comprising aa sensor head and a sensor readout device, wherein the sensor head and the partially coherent light source are arranged such that the sensor head and the partially coherent light source define a sample space therebetween for receiving a substrate having a sample, wherein
an optical image of a sample in a sample chamber through the partially coherent light source onto the sensor head represents an intensity pattern.

2. The device of claim 1, wherein the partially coherent light source and the sensor head are arranged to be set up for lensless imaging, wherein no lens and/or no optical component affecting optical coherence of light of the partially coherent light source as well as light guidance is/are arranged between the partially coherent light source and the image sensor.

3. The device according to claim 1, wherein the partially coherent light source has a coherence length between 1 and 100 μm.

4. The device according to claim 1, wherein the sensor readout device is spaced apart from the sensor head such that, during operation of the sensor readout device, the sample space is substantially not heated by the operation.

5. The device according to claim 1, further comprising one or more filters arranged between the partially coherent light source and the sensor head, wherein at least one of the filters is directly applied to the sensor head, in particular is directly vapor-deposited.

6. The device according to claim 1, further comprising a support member adapted to support a sample and adapted to receive the sensor head.

7. The device according to claim 6, wherein the support member comprises a first portion for receiving the sensor head and a second portion, wherein thermal conductivity and/or thermal capacity and/or emissivity of the second portion is determined to be substantially identical to thermal conductivity and/or thermal capacity and/or emissivity of the first portion after receiving the sensor head.

8. The device according to one of claim 6, wherein a surface of a support element is adapted for positioning of sample containers, in particular by having a profile which is adapted to a shape of sample containers.

9. The device according to claim 1, wherein the sensor head and/or the partially coherent light source are adapted to be movable relative to a sample placeable in the sample space.

10. The device according to claim 1, wherein the device is a first device and where in the first device is adapted to be placed on top of a second device similar to the first device, such that the first device cannot be displaced relative to or fall off of the second device.

11. A system for monitoring samples, in particular biological cells, the system comprising:
one or more devices, each device including:
a partially coherent light source; and
an image sensor including a sensor head and a sensor readout device, wherein the sensor head and the partially coherent light source are arranged such that the sensor head and the partially coherent light source define a sample space therebetween for receiving a substrate having a sample, wherein an optical image of a sample in a sample chamber through the partially coherent light source onto the sensor head represents an intensity pattern;
wherein the system is adapted to allow a plurality of samples to be monitored by the one or more devices.

12. The system according to claim 11, wherein monitoring comprises moving the one or more devices and/or moving the samples.
